(19) United States
(12) Patent Application Publication
(10) Pub. No.: US 2021/0098084 A1
(43) Pub. Date: Apr. 1, 2021

(54) Method and System for Material Screening

(71) Applicants: Nissan North America, Inc., Franklin, TN (US); United States of America as Represented by the Administrator of NASA, Washington, DC (US)

(72) Inventors: Akiyoshi Park, Tokyo (JP); Taehee Han, West Bloomfield, MI (US); Shigemasa Kuwata, Palo Alto, CA (US); Maarten Sierhuis, San Francisco, CA (US); Xin Yang, East Palo Alto, CA (US); Atsushi Ohma, Kanagawa (JP); Balachandran Gadaguntla Radhakrishnan, San Mateo, CA (US); Shreyas Honrao, Sunnyvale, CA (US); John Lawson, San Francisco, CA (US); Najamuddin Mirza Baig, San Jose, CA (US); Mohit Rakesh Mehta, Santa Clara, CA (US)

(21) Appl. No.: 16/587,937

(22) Filed: Sep. 30, 2019

(51) Int. Cl. G16C 60/00; G06N 20/20; G06F 16/9035; G16C 20/70

(52) U.S. Cl. CPC G16C 60/00; G16C 20/70; G06F 16/9035; G06N 20/20

(57) ABSTRACT

A method for screening materials may include obtaining materials from a database. The method may include screening the materials to obtain a one or more screened materials. The method may include generating a training set based on the screened materials, validated experimental data, or both. The method may include establishing a machine learning screening model based on the training set, one or more target parameters, or both. The method may include applying the machine learning screening model to uncharacterized materials. The method may include outputting one or more materials having characteristics matching the target parameters.

### TECHNICAL FIELD

This disclosure relates to material screening methods and systems.

### BACKGROUND

Typical approaches for developing compounds may be time-consuming and costly. Manual screening of materials is impractical. Accordingly, methods and systems are needed for material screening to time and costs.

### SUMMARY

Disclosed herein are aspects, features, elements, implementations, and embodiments of systems and methods for material screening.

In an aspect, a method may include obtaining materials from a database. The method may include screening the materials to obtain a one or more screened materials. The method may include generating a training set based on the screened materials, validated experimental data, or both. The method may include establishing a machine learning screening model based on the training set, one or more target parameters, or both. The method may include applying the machine learning screening model to uncharacterized materials. The method may include outputting one or more materials having characteristics matching the target parameters.

In an aspect, a method may include establishing a machine learning screening model. The machine learning screening model may be based on a training set, one or more target parameters, or both. The training set may be based on a plurality of screened materials, validated experimental data, or both. The method may include applying the machine learning screening model to uncharacterized materials. The method may include outputting one or more materials having characteristics matching the target parameters. The method may include updating the machine learning screening model based on validated experimental data of the one or more materials having characteristics matching the target parameters.

In one or more aspects, the screening of the materials may include constructing a canonical phase diagram for each of the materials. In one or more aspects, the method may include computing an electrochemical stability for each material. The calculation of the electrochemical stability may be based on a respective canonical phase diagram. In one or more aspects, the method may include filtering the materials. The materials may be filtered based on a target electrochemical stability range. The filtering may result in obtaining one or more pre-screened materials. The one or more pre-screened materials may be filtered for oxides, halides, or nitrides to obtain one or more screened materials.

One or more aspects may include computing an ionic conductivity for each material. The ionic conductivity may be based on text mining, manual search, or both. Computing the ionic conductivity may be based on an activation energy calculation. One or more aspects may include computing a dendrite suppression value for each material. One or more aspects may include computing a thickness for each material. In one or more aspects, the machine learning screening model may be a linear regression model, a random forest model, or an Xgboost model.

Variations in these and other aspects, features, elements, implementations, and embodiments of the methods, apparatuses, procedures, and algorithms disclosed herein are described in further detail hereinafter.

### BRIEF DESCRIPTION OF THE DRAWINGS

The various aspects of the methods and apparatuses disclosed herein will become more apparent by referring to the examples provided in the following description and drawings in which:

FIG. 1 is a diagram of an example of a system for material design;

FIG. 2 is a flow diagram of an example of a material design method;

FIG. 3 is a diagram of an example of a screening model;

FIG. 4 is a flow diagram of an example of a training method for a machine learning screening model;

FIG. 5 is a flow diagram of an example of an experimental validation method; and

FIG. 6 is a flow diagram of an example of a material design method for a passivation layer of an all solid state battery.

### DETAILED DESCRIPTION

As used herein, the terminology “computer” or “computing device” includes any unit, or combination of units, capable of performing any method, or any portion or portions thereof, disclosed herein.

As used herein, the terminology “processor” indicates one or more processors, such as one or more special-purpose processors, one or more digital signal processors, one or more microprocessors, one or more controllers, one or more microcontrollers, one or more application processors, one or more Application Specific Integrated Circuits, one or more Application Specific Standard Products, one or more Field Programmable Gate Arrays, any other type or combination of integrated circuits, one or more state machines, or any combination thereof.

As used herein, the terminology “memory” indicates any computer-usable or computer-readable medium or device that can tangibly contain, store, communicate, or transport any signal or information that may be used by or in connection with any processor. For example, a memory may be one or more read-only memories (ROM), one or more random-access memories (RAM), one or more registers, one or more low power double data rate (LPDDR) memories, one or more cache memories, one or more semiconductor memory devices, one or more magnetic media, one or more optical media, one or more magneto-optical media, or any combination thereof.

As used herein, the terminology “instructions” may include directions or expressions for performing any method, or any portion or portions thereof, disclosed herein, and may be realized in hardware, software, or any combination thereof. For example, instructions may be implemented as information, such as a computer program, stored in memory that may be executed by a processor to perform any of the respective methods, algorithms, aspects, or combinations thereof, as described herein. Instructions, or a portion thereof, may be implemented as a special-purpose processor, or circuitry, that may include specialized hardware for carrying out any of the methods, algorithms, aspects, or combinations thereof, as described herein. In some implementations, portions of the instructions may be distributed across multiple processors on a single device, or across multiple processors on multiple devices that may communicate directly or across a network, such as a local area network, a wide area network, the Internet, or a combination thereof.

As used herein, the terminology “example,” “embodiment,” “implementation,” “aspect,” “feature,” or “element” indicates serving as an example, instance, or illustration. Unless expressly indicated otherwise, any example, embodiment, implementation, aspect, feature, or element is independent of each other example, embodiment, implementation, aspect, feature, or element and may be used in combination with any other example, embodiment, implementation, aspect, feature, or element.

As used herein, the terminology “determine” and “identify,” or any variations thereof, includes selecting, ascertaining, computing, looking up, receiving, determining, establishing, obtaining, or otherwise identifying or determining in any manner whatsoever using one or more of the devices shown and described herein.

As used herein, the terminology “or” is intended to mean an inclusive “or” rather than an exclusive “or.” That is, unless specified otherwise or clearly indicated otherwise by the context, “X includes A or B” is intended to indicate any of the natural inclusive permutations thereof. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from the context to be directed to a singular form.

Further, for simplicity of explanation, although the figures and descriptions herein may include sequences or series of operations or stages, elements of the methods disclosed herein may occur in various orders or concurrently. Additionally, elements of the methods disclosed herein may occur with other elements not explicitly presented and described herein. Furthermore, not all elements of the methods described herein may be required to implement a method in accordance with this disclosure. Although aspects, features, and elements are described herein in particular combinations, each aspect, feature, or element may be used independently or in various combinations with or without other aspects, features, and elements.

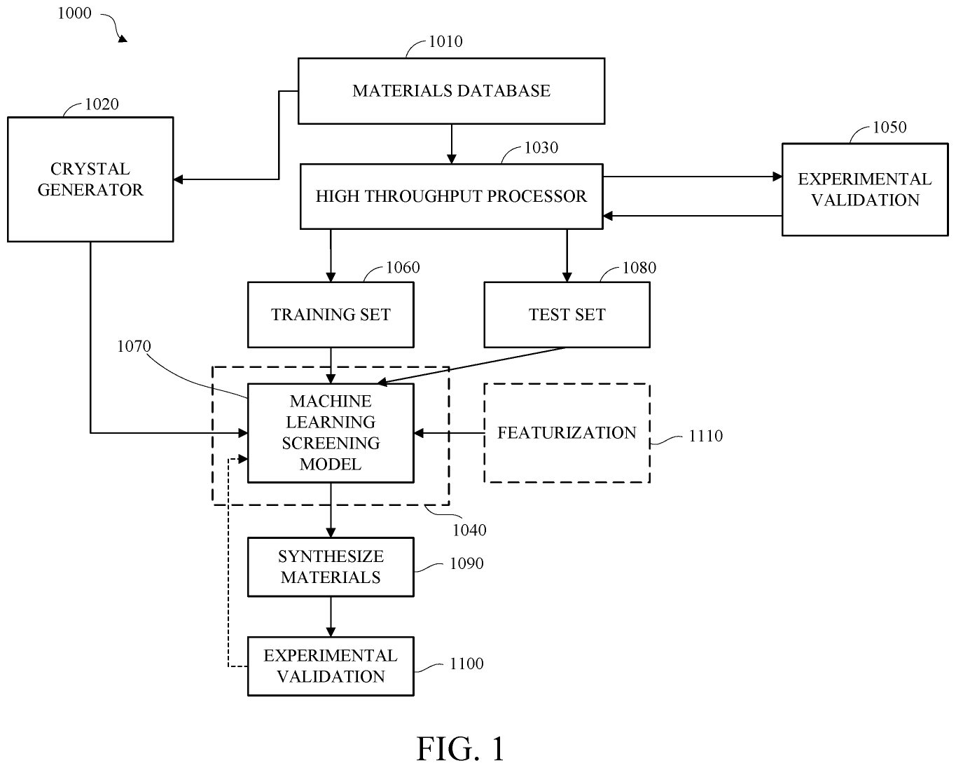

FIG. 1 is a diagram of an example of a system 1000 for material design. The system 1000 may include a materials database 1010, a crystal generator 1020, and one or more processors, for example high throughput processor 1030 and processor 1040. Although shown separately in FIG. 1, in some implementations, processor 1040 may be a sub-processor and combined with high throughput processor 1030.

The materials database 1010 may be any type of database configured to store data associated with a large number of materials, for example 10¹⁰ or more materials. The data associated with the materials may be crystal structure data. The materials database 1010 may include data for each material that includes, but is not limited to, crystal volume, number of nsites, point group, band gap, density, energy (E) above hull, Fermi E, E per atom, and formation E per atom. The materials database 1010 may also include values for ionic conductivity, electronic conductivity, stability, cost, dendrite suppression, or any combination thereof.

The materials database 1010 may reside on a memory. The memory may include any tangible non-transitory computer-usable or computer-readable medium capable of, for example, containing, storing, communicating, or transporting machine readable instructions, or any information associated therewith, for use by or in connection with a processor, for example high throughput processor 1030. The memory may be, for example, one or more solid-state drives, one or more memory cards, one or more removable media, one or more read-only memories, one or more random-access memories, one or more disks (including a hard disk, a floppy disk, an optical disk), a magnetic or optical card, or any type of non-transitory media suitable for storing electronic information, or any combination thereof.

As shown in FIG. 1, the high throughput processor 1030 is configured to obtain the data associated with the materials from the materials database 1010 and screen the obtained materials. For example, the high throughput processor 1030 may be configured to screen for electrochemical stability, ionic stability, or both. In examples where the materials are screened for both electrochemical stability and ionic stability, the screening may be performed in parallel or series. In some embodiments, the high throughput processor 1030 may be configured to compute values for ionic conductivity, electronic conductivity, stability, cost, dendrite suppression, or any combination thereof.

In an example where the target window range for electrochemical stability is 0-1.72 V (vs. Li/Li+), the high throughput processor 1030 may obtain over 19,000 Li containing compounds from the materials database 1010. The high throughput processor 1030 may filter the obtained materials for Li containing compounds with a >1 eV band gap to reduce the number of compounds of interest to 8891. The 8891 compounds of interest may be further classified into a subspace of the number of elements as shown in Table 1 below.

TABLE 1

| Subspace | Number of Compounds |
|---|---|
| 3 element | 52 |
| 4 element | 517 |
| 5 element | 408 |
| 6 element | 109 |
| 7 element | 8 |
| 8 element | 1 |

As shown from Table 1 above, the classification by the above subspaces further reduces the number of compounds of interest to 1095. The high throughput processor 1030 may be configured to construct a grand canonical phase diagram, also known as a grand canonical ensemble or microcanonical ensemble, for each of the 1095 compounds of interest. The high throughput processor 1030 may be configured to compute a value, for each compound of interest, representing the electrochemical stability, the ionic conductivity, or both. The electrochemical stability of each compound may be based on the respective grand canonical phase diagram. The high throughput processor 1030 may be configured to filter the compounds of interest based on a target electrochemical stability range to obtain one or more pre-screened materials. The high throughput processor 1030 is configured to filter the pre-screened materials for one or more other parameters, to obtain one or more screened materials. The one or more screened materials are subject for experimental validation 1050, and the validated screened materials may be used as a training set 1060 to train the machine learning screening model 1070.

A test set 1080 may be output from the high throughput processor 1030 and input to the machine learning screening model 1070. The test set 1080 may include any number of compounds of interest. The machine learning screening model 1070 is configured to output one or more materials for experimental validation. Each of the one or more materials for experimental validation is synthesized 1090 for experimental validation 1100. The results of the experimental validation may be fed back into the machine learning screening model 1070. If needed, the machine learning screening model 1070 may be updated based on the results of the experimental validation.

In some implementations, featurization 1110 may be performed and used to train the machine learning screening model 1070. Featurization 1110 may be used to create features from raw data to help facilitate the machine learning process and increase the predictive power of the machine learning algorithms. Multiple models can be used, such as normalization, binning and PCA. Some of the features used across the models include the element property and the band center.

The crystal generator 1020 may be configured to generate novel crystals for screening with the machine learning screening model 1070. The machine learning screening model 1070 may be based on a linear regression model, a random forest model, an Xgboost model, or any other suitable model. The crystal generator 1020 may obtain crystal structures from the materials database 1010 and substitute one or more elements of the obtained crystal structures to generate theoretical crystal structures that are not present in the materials database 1010. The crystal generator 1020 is configured to input the theoretical crystal structures into the machine learning screening model 1070. The theoretical crystal structures may include any number of compounds of interest. The machine learning screening model 1070 is configured to output one or more theoretical crystal structures for experimental validation that may satisfy the desired properties. Each of the one or more theoretical crystal structures for experimental validation is synthesized 1090 for experimental validation 1100. The results of the experimental validation may be input back into the machine learning screening model 1070. If needed, the machine learning screening model 1070 may be updated based on the results of the experimental validation.

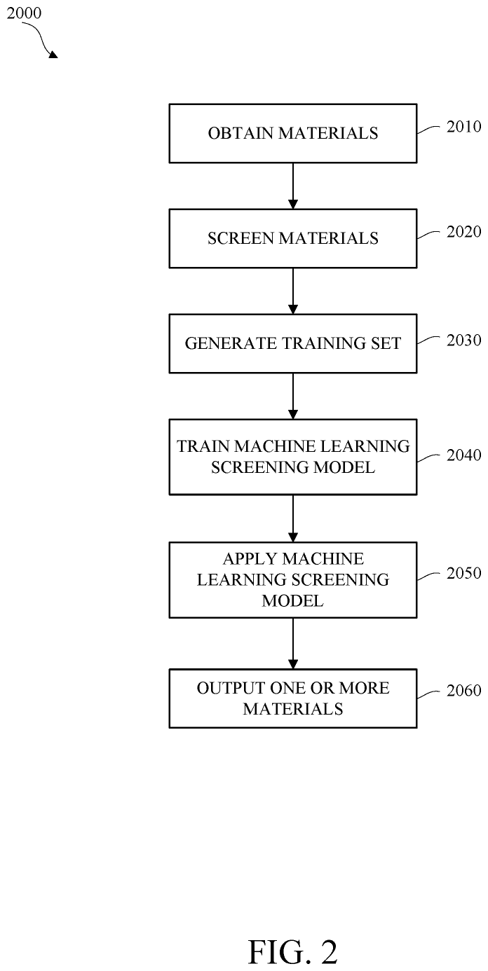

FIG. 2 is a flow diagram of an example of a material design method 2000. Referring to FIG. 2, the method 2000 includes obtaining materials 2010. Obtaining materials 2010 may include obtaining the data associated with the materials from the materials database 1010 of FIG. 1.

The method 2000 includes screening the obtained materials 2020. For example, screening the obtained materials 2020 may include screening for electrochemical stability, ionic stability, or both. In examples where the materials are screened for both electrochemical stability and ionic stability, the screening may be performed in parallel or series.

The method 2000 includes generating a training set 2030. Generating a training set 2030 may include constructing a grand canonical phase diagram for each compound of interest. Generating a training set 2030 may include computing, for each compound of interest, the electrochemical stability, the ionic conductivity, or both. The electrochemical stability of each compound may be based on the respective grand canonical phase diagram. Generating a training set 2030 may include filtering the compounds of interest based on a target electrochemical stability range, or any other parameter alone or in combination, to obtain one or more pre-screened materials. Generating a training set 2030 may include filtering the pre-screened materials for one or more other parameters, to obtain one or more screened materials. The one or more screened materials are subject for experimental validation, and the validated screened materials may output as a training set to train a machine learning screening model 2040.

The method 2000 includes applying the machine learning screening model 2050 to theoretical crystal structures. The theoretical crystal structures may include any number of compounds of interest. By applying the machine learning screening model 2050 to the theoretical structures, the system may output one or more materials 2060 that meet the desired criteria for experimental validation.

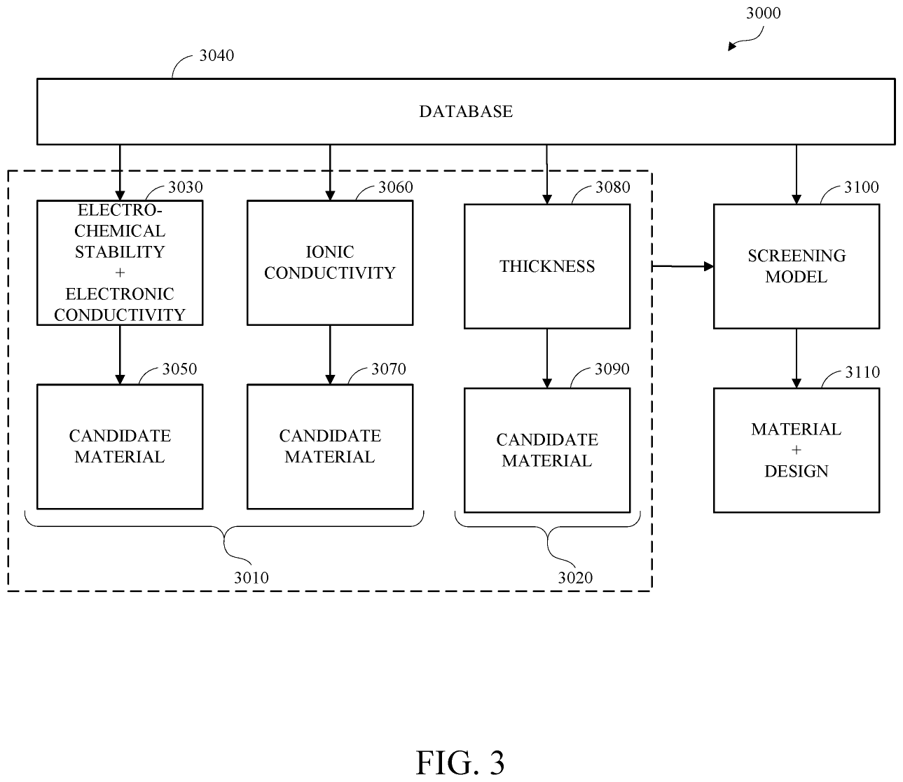

FIG. 3 is a diagram of an example of a screening model 3000. As shown in FIG. 3, the screening model 3000 includes a material screening portion 3010 and a design screening portion 3020. The material screening portion 3010 may include calculating the electrochemical stability and the electronic conductivity 3030 of one or more materials from a database 3040 and identifying one or more candidate materials 3050. The one or more candidate materials 3050 may be identified based on the electrochemical stability, electronic conductivity, or both. The database 3040 may be any database, for example materials database 1010 of FIG. 1. The material screening portion 3010 may include calculating the ionic conductivity 3060 of one or more materials from the database 3040 and identifying one or more candidate materials 3070. The design screening portion 3020 may include calculating the thickness 3080 of one or more materials from the database 3040 and identifying one or more candidate materials 3090. Each calculated value and identified candidate material in the material screening portion 3010 and the design screening portion 3020 may be combined in series or in parallel to form the screening model 3100. The screening model 3100 may then be used to identify 3110 an optimal material and design based on one or more parameters or material properties.

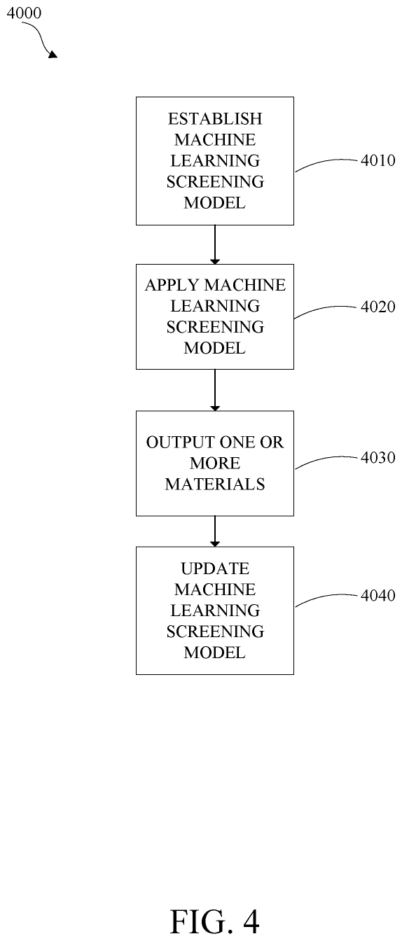

FIG. 4 is a flow diagram of an example of a training method 4000 for a machine learning screening model. Referring to FIG. 4, the method 4000 includes establishing a machine learning screening model 4010. The machine learning screening model may be a linear regression model, a random forest model, an Xgboost model, or any other suitable model. The machine learning screening model may be based on one or more material parameters. The one or more desired material parameters may include, for example, electrochemical stability, ionic conductivity, porosity, thickness, cost, dendrite suppression, crystal volume, point group, nsites, band gap, density, E above hull, Fermi E, E per atom, formation E/atom, or any combination thereof. The method 4000 includes applying the machine learning screening model 4020 to theoretical crystal structures. The theoretical crystal structures may include any number of compounds of interest. By applying the machine learning screening model 4020 to the theoretical structures, the system may output one or more materials 4030 that meet the desired criteria for experimental validation. The method 4000 may include updating the machine learning model 4040. The machine learning model may be updated based on the experimental validation results. For example, compounds that are not confirmed via experimental validation may be removed from the machine learning model. The machine learning model may then automatically adapt to identify compounds that may have similar characteristics as a compound that was not confirmed via experimental validation, identify trends in such compounds, and automatically remove such compounds from a list of compounds of interest.

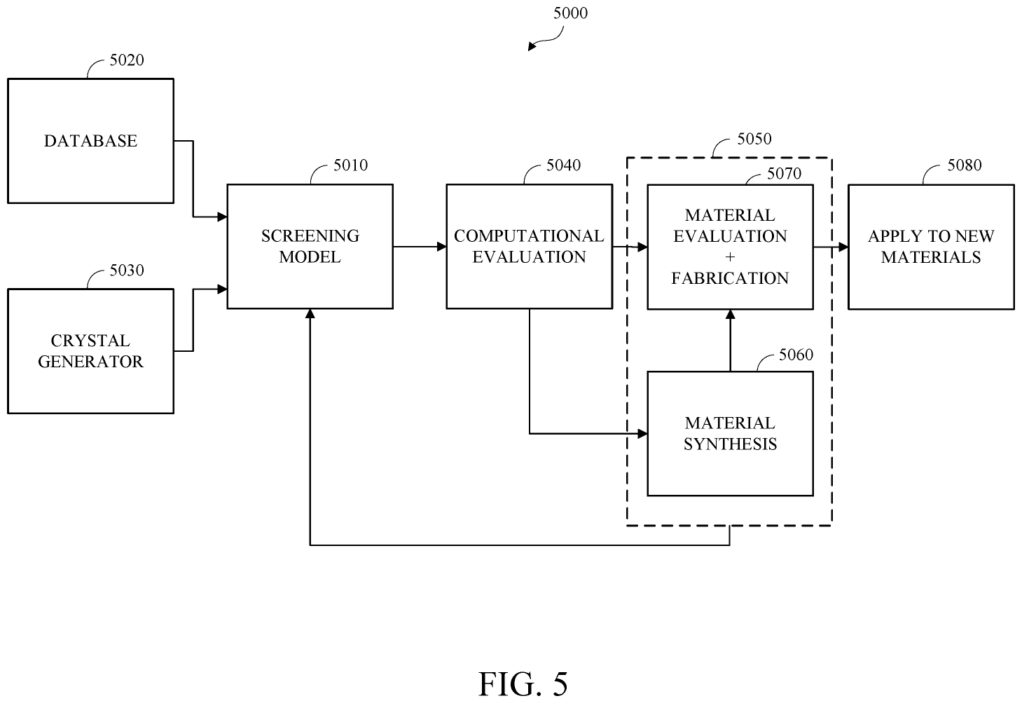

FIG. 5 is a flow diagram of an example of an experimental validation method 5000. Referring to FIG. 5, the method 5000 includes developing a screening model 5010 based on existing materials in database 5020. For example, the materials obtained from database 5020 may be screened for electrochemical stability, ionic stability, or both, to develop the screening model 5010. In examples where the materials are screened for both electrochemical stability and ionic stability, the screening may be performed in parallel or series.

The crystal generator 5030 may be configured to generate novel crystals for screening with the screening model 5010. The crystal generator 5030 may obtain crystal structures from the database 5020 and substitute one or more elements of the obtained crystal structures to generate theoretical crystal structures that are not present in the database 5020. The crystal generator 5030 is configured to input the theoretical crystal structures into the screening model 5010. The theoretical crystal structures may include any number of compounds of interest. The screening model 5010 is used to perform a computational evaluation 5040 to predict one or more chemical properties of the theoretical crystal structures. The result of the computational evaluation 5040 is an output of one or more theoretical crystal structures for experimental validation 5050 that have predicted chemical properties that match desired chemical properties.

Experimental validation 5050 includes synthesizing 5060 the theoretical crystal structures that have predicted chemical properties that match desired chemical properties. The synthesized theoretical structures may then be experimentally evaluated 5070 to confirm whether the predicted chemical properties match the actual desired chemical properties. In some examples, the experimental evaluation may include the fabrication of a product using the synthesized theoretical structure. Results of the experimental validation 5050 may be input to the screening model 5010 and the method 5000 may be repeated and applied to new materials 5080.

Typical solid state batteries include a Li₁₀GeP₂S₁₂ (LGPS) solid state electrolyte (SE) layer disposed on a Li film, and a sulfur layer disposed on the LGPS layer. The sulfur layer may be a cathode layer and the Li film may be an anode. The Li film may also be referred to as the Li metal layer. LGPS exhibits thermodynamically a narrow electrochemical window, despite a high ionic conductivity (12 mS/cm). The thermodynamic stability of LGPS ranges from approximately 2 to 2.3 V (vs. Li/Li+).

In some embodiments, an indium thin film may be disposed between the Li film and the LGPS layer to compensate for the narrow electrochemical window of the LGPS. In some embodiments, an passivation layer may be disposed between the Li film and the LGPS layer to compensate for the narrow electrochemical window of the LGPS. The passivation layer may exhibit SE-like properties. The passivation layer may be used to maximize the energy density and durability of the Li—S all solid state battery (ASSB). A material science based approach may be used to develop the passivation layer materials between the Li metal and the LGPS layer.

To find an ideal passivation material, a data driven screening, a computational validation, and an experimental validation may be performed in a repetitive manner. The data driven screening may use machine learning to predict one or more features of a material that may be ideal for a passivation layer. Computational validation may be used to validate the performance of the material. Experimental validation may be used to synthesize the materials and fabricate ASSB cells to experimentally evaluate performance. Data from the computational validation, experimental validation, or both may be input to the screening model.

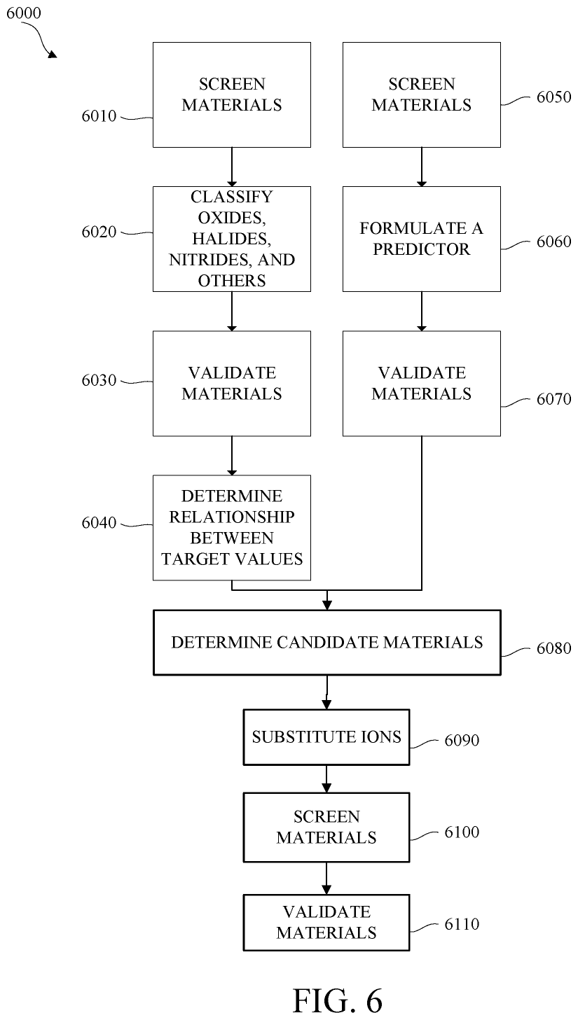

FIG. 6 is a flow diagram of an example of a material design method 6000 for a passivation layer of an all solid state battery. In this example, the method 6000 includes screening materials 6010 based on electrochemical stability. Screening materials 6010 may include obtaining a list of compounds of interest that contain Li from a database, such as materials database 1010 of FIG. 1. In this example, the database may include greater than 130,000 compounds, and of those compounds, approximately 9,000 compounds may contain Li.

A processor, such as the high throughput processor 1030 of FIG. 1, may be configured to filter the compounds of interest based on a target electrochemical stability range to obtain one or more pre-screened materials. In this example, the screening criteria used included a lower bound stability window of 0 V, an upper bound stability window of 1.5 V, and a band gap of greater than 1.0 eV. Using this screening criteria, the number of compounds with chemistries that have some stability range against Li is 732, and the number of compounds with chemistries that have some stability range against Li metal is 86. The processor may be configured to classify 6020 the pre-screened materials for oxides, halides, nitrides, others that do not contain rare earth elements, or any combination thereof, to obtain one or more screened materials. In this example, the number of screened materials subject for experimental validation may be 10 to 15.

The method 6000 includes validating the screened materials 6030. Validating the screened materials 6030 may include synthesizing the screened materials that have predicted chemical properties that match desired chemical properties. The synthesized screened materials may then be experimentally evaluated to confirm whether the predicted chemical properties match the actual desired chemical properties. The method 6000 may include determining 6040 a relationship between two or more target values. For example, a minimum and maximum of an electrochemical stability window may be plotted on a graph to determine their relationship. In an example, a cluster may be chosen where the minimum of the electrochemical stability window of each candidate material is zero and the maximum of the electrochemical stability window of each candidate material is 1.72. In some implementations, graph neural networks may be used as an alternative to make a descriptor-less machine learning method to determine electrochemical stability. An example of a neural network is Megnet.

The method 6000 includes screening materials 6050 for ionic conductivity. The screening of materials 6050 for ionic conductivity may be done in parallel with the screening of materials 6010 for electrochemical stability or in series. Screening materials 6050 for ionic conductivity may include using text mining, performing a manual search, or both, to compile the ionic conductivities of approximately 100 materials. Alternatively, screening materials 6050 for ionic conductivity may include calculating an activation energy of approximately 100 candidate materials.

The method 6000 includes formulating a predictor 6060 using machine learning. The predictor may be used to obtain one or more screened materials. As an example, the predictor can be a Machine Learning Model (ML) that provides the value for electrochemical conductivity (EC) of the material. If the EC conductivity is within the recommended range, this indicates that the material is a good candidate for the passivation layer of the battery

The method 6000 includes validating the screened materials 6070. Validating the screened materials 6070 may include synthesizing the screened materials that have predicted chemical properties that match desired chemical properties. The synthesized screened materials may then be experimentally evaluated to confirm whether the predicted chemical properties match the actual desired chemical properties.

The method 6000 includes determining candidate materials 6080. The validated materials from the electrochemical stability screening and the ionic conductivity screening may be used in determining the candidate materials 6080. A crystal generator, such as the crystal generator 5030 of FIG. 5 may be configured to generate novel crystals for screening. The crystal generator may obtain the determined candidate materials and substitute one or more ions 6090 of the obtained candidate materials to generate theoretical crystal structures. The theoretical crystal structures may include any number of compounds of interest. The method includes screening the compounds of interest 6100 by performing a computational evaluation to predict one or more chemical properties of the theoretical crystal structures. The result of the computational evaluation is an output of one or more theoretical crystal structures for experimental validation 6110 that have predicted chemical properties that match desired chemical properties.

Experimental validation 6110 includes synthesizing the theoretical crystal structures that have predicted chemical properties that match desired chemical properties. The synthesized theoretical structures may then be experimentally evaluated to confirm whether the predicted chemical properties match the actual desired chemical properties.

Experimental validation 6110 may include performing cyclic voltammetry. For example, a reference electrode such as an indium foil may be disposed between the Li film and the passivation layer to measure the Li/Li+ potential. In some embodiments, a semi-blocking configuration may be used. In the semi-blocking configuration, an additional layer may be disposed on the passivation layer. The additional layer may include the passivation layer material and carbon in order to induce a higher surface area. The additional layer may be disposed between the passivation layer and a copper plate that functions as a counter electrode. Typical semi-blocking configurations have undesirable interfacial contact between the blocking surface that makes it impossible to see the effect of decomposition. The additional layer that includes the passivation layer material and carbon may be used to see the decomposition in the additional layer.

In the embodiments described herein, a processor may include any device or combination of devices, now-existing or hereafter developed, capable of manipulating or processing a signal or other information, including optical processors, quantum processors, molecular processors, or a combination thereof. For example, the processor may include one or more special-purpose processors, one or more digital signal processors, one or more microprocessors, one or more controllers, one or more microcontrollers, one or more integrated circuits, one or more Application Specific Integrated Circuits, one or more Field Programmable Gate Arrays, one or more programmable logic arrays, one or more programmable logic controllers, one or more state machines, or any combination thereof. The processor 1330 may be operatively coupled with a memory, an electronic communication interface, an electronic communication unit, a user interface, a sensor, or any combination thereof. For example, the processor may be operatively coupled with the memory via a communication bus.

The memory may include any tangible non-transitory computer-usable or computer-readable medium capable of, for example, containing, storing, communicating, or transporting machine readable instructions, or any information associated therewith, for use by or in connection with the processor. The memory may be, for example, one or more solid-state drives, one or more memory cards, one or more removable media, one or more read-only memories, one or more random-access memories, one or more disks (including a hard disk, a floppy disk, an optical disk), a magnetic or optical card, or any type of non-transitory media suitable for storing electronic information, or any combination thereof.

The above-described aspects, examples, and implementations have been described in order to facilitate easy understanding of the disclosure and are not limiting. On the contrary, the disclosure covers various modifications and equivalent arrangements included within the scope of the appended claims, which scope is to be accorded the broadest interpretation as is permitted under the law so as to encompass all such modifications and equivalent arrangements.

What is claimed is:

1. A method comprising:
obtaining a plurality of materials from a database;
screening the plurality of materials to obtain a plurality of screened materials;
generating a training set based on the plurality of screened materials and validated experimental data;
establishing a machine learning screening model based on the training set and target parameters;
applying the machine learning screening model to uncharacterized materials; and
outputting one or more materials having characteristics matching the target parameters.

2. The method of claim 1, wherein screening the plurality of materials comprises constructing a canonical phase diagram for each of the plurality of materials.

3. The method of claim 2, further comprising:
computing an electrochemical stability for each of the plurality of materials based on a respective canonical phase diagram.

4. The method of claim 3, further comprising:
filtering the plurality of materials based on a target electrochemical stability range to obtain a plurality of pre-screened materials.

5. The method of claim 4, further comprising:
filtering the plurality of pre-screened materials for oxides, halides, or nitrides to obtain the plurality of screened materials.

6. The method of claim 1, further comprising:
computing an ionic conductivity for each of the plurality of materials.

7. The method of claim 6, wherein computing the ionic conductivity is based on text mining and a manual search.

8. The method of claim 6, wherein computing the ionic conductivity is based on an activation energy calculation.

9. The method of claim 1, further comprising:
computing a dendrite suppression value for each of the plurality of materials.

10. The method of claim 1, further comprising:
computing a thickness for each of the plurality of materials.

11. The method of claim 1, wherein the machine learning screening model is a linear regression model, a random forest model, or an Xgboost model.

12. A method comprising:
establishing a machine learning screening model based on a training set and target parameters, wherein the training set is based on a plurality of screened materials and validated experimental data;
applying the machine learning screening model to uncharacterized materials;
outputting one or more materials having characteristics matching the target parameters; and
updating the machine learning screening model based on validated experimental data of the one or more materials having characteristics matching the target parameters.

13. The method of claim 12, further comprising:
computing an electrochemical stability for each of the one or more materials having characteristics matching the target parameters.

14. The method of claim 13, wherein computing the electrochemical stability is based on a canonical phase diagram.

15. The method of claim 12, further comprising:
computing an ionic conductivity for each of the one or more materials having characteristics matching the target parameters.

16. The method of claim 15, wherein computing the ionic conductivity is based on text mining and a manual search.

17. The method of claim 15, wherein computing the ionic conductivity is based on an activation energy calculation.

18. The method of claim 12, further comprising:
computing a dendrite suppression value for each of the one or more materials having characteristics matching the target parameters.

19. The method of claim 12, further comprising:
computing a thickness for each of the one or more materials having characteristics matching the target parameters.

20. The method of claim 12, wherein the machine learning screening model is a linear regression model, a random forest model, or an Xgboost model.
